(19) United States
(12) Patent Application Publication
(10) Pub. No.: US 2021/0121491 A1
(43) Pub. Date: Apr. 29, 2021

(54) AGI-134 COMBINED WITH A CHECKPOINT INHIBITOR FOR THE TREATMENT OF SOLID TUMORS

(71) Applicant: BioLineRx Ltd., ModiIn (IL)

(72) Inventor: Irit Carmi LEVY, Jerusalem (IL)

(73) Assignee: BioLineRx Ltd., ModiIn (IL)

(21) Appl. No.: 17/056,803

(22) PCT Filed: May 27, 2019
(86) PCT No.: PCT/IL2019/050601

§ 371 Date: Nov. 19, 2020

Related U.S. Application Data
(60) Provisional application No. 62/676,999, filed on May 27, 2018.

(51) Int. Cl. A61K 31/7032; C07K 16/28

(52) U.S. Cl. CPC A61K 31/7032; C07K 16/2818

(57) ABSTRACT

A method of treating a tumor in a subject is disclosed. The method comprises administering to the subject a therapeutically effective amount of:
i) Pembrolizumab; and
ii) a lipid having a structural formula as illustrated in FIG. 1 (AGI-134).

### RELATED APPLICATION

This application claims the benefit of priority from U.S. Provisional Patent Application No. 62/676,999 filed on 27 May 2018, the contents of which are incorporated herein by reference in their entirety.

### FIELD AND BACKGROUND OF THE INVENTION

The present invention, in some embodiments thereof, relates to a combination therapy for treatment of cancer.

The major cause of death in cancer patients with solid tumors is the recurrence of the cancer after surgery as multiple metastases are non-respectable and/or refractory to any therapy. The majority of these patients are considered to have a terminal cancer disease. As no treatment is available for them, many of these patients die within weeks or a few months after detection of metastatic tumor lesions.

Tumors develop in cancer patients because the immune system fails to detect tumor cells as cells that ought to be destroyed. Tumor cells express tumor antigens in a large proportion of cancer patients. These patient-specific tumor antigens may elicit a protective anti-tumor immune response. Tumor cells, or tumor cell membranes, have to be internalized by antigen presenting cells in order to induce the development of an anti-tumor immune response. However, the immune system in cancer patients displays “ignorance” toward the tumor antigens that is associated with early development of the tumor in a “stealthy” way, so it is “invisible” to antigen presenting cells (Pardoll D M. Clin. Immunol. 2000; 95:S44-49; and Dunn G P et al. Nat Immunol 2002; 3: 991-8).

In addition, the tumor microenvironment and local cytokine milieu are often suppressive toward immune function and can actively induce immune cell anergy and death (Malmberg K J. Cancer Immunol. Immunother. 2004; 53: 879-92; Lugade A A et al. J. Immunol. 2005; 174: 7516-23). Effective treatment of such metastatic tumor lesions requires two components:

1. Destruction of the lesions that are large enough to be detected visually or by imaging technology, and

2. Induction of a protective anti-tumor immune response against tumor antigens.

Such an immune response results in immune-mediated detection, regression, and/or destruction of micrometastases which cannot be detected visually and are not detectable by imaging.

Induction of a protective anti-tumor immune response requires uptake of the tumor cells or cell membranes by antigen presenting cells and their transportation to the draining lymph nodes, where the antigen presenting cells process the tumor antigen molecules. The majority of these tumor antigens are specific to the individual patient. The immunogenic tumor antigen peptides are presented by antigen presenting cells in association with class I or class II MHC molecules for the activation of tumor specific CD8⁺ and CD4⁺T cells, respectively. Only after these T cells are activated by the processed and presented tumor antigen peptides, can these lymphocytes proliferate, leave the lymph nodes, circulate in the body, seek and destroy metastatic tumor cells expressing tumor antigens. In addition, though only after they are activated, helper T cells can provide help to B cells for producing antibodies against the tumor antigens. However, since the tumor cells naturally evolve to be “invisible” to antigen presenting cells, the developing tumor metastases are usually ignored by the immune system to the extent that metastasizing tumor cells can proliferate even within lymph nodes. Therefore, eliciting an effective anti-tumor immune response requires effective targeting of tumor cells to antigen presenting cells.

US 2006251661 describes methods of administering natural glycolipid compounds to tumor lesions that induce local expression of α-Gal epitopes within the tumor which interact with the natural anti-Gal antibody.

US 20170266214 discloses a glycolipid composition that inserts into tumor cell membranes so as to elicit a protective immune response in the host against tumor cells expressing the tumor antigens.

### SUMMARY OF THE INVENTION

According to an aspect of some embodiments of the present invention there is provided a method of treating a tumor in a subject, comprising administering to the subject a therapeutically effective amount of:

i) Pembrolizumab; and

ii) a lipid having a structural formula as illustrated in FIG. 1 (AGI-134);

thereby treating the tumor in the subject.

According to an aspect of some embodiments of the present invention there is provided a method of treating a tumor in a subject, comprising:

i) intratumorally administering to the subject between 25-200 mg of AGI-134;

ii) intravenously administering to the subject 200 mg of Pembrolizumab; wherein said Pembrolizumab and said AGI-134 are administered not more than two hours apart, wherein said Pembrolizumab and said AGI-134 are administered once every three weeks for a total of four cycles; and subsequently

(iii) intravenously administering to the subject 200 mg of Pembrolizumab once every three weeks for up to one year in the absence of said AGI-134, thereby treating the tumor in the subject.

According to an aspect of some embodiments of the present invention there is provided Pembrolizumab and AGI-134 for use in treating a tumor in a subject.

According to some embodiments of the invention, the Pembrolizumab and said AGI-134 are in separate formulations.

According to some embodiments of the invention, the Pembrolizumab is administered following administration of said AGI-134.

According to some embodiments of the invention, the Pembrolizumab and said AGI-134 are administered not more than two hours apart.

According to some embodiments of the invention, the method further comprises administering to said subject Pembrolizumab in the absence of said AGI-134 following said administering of Pembrolizumab and said AGI-134.

According to some embodiments of the invention, the Pembrolizumab is administered intravenously.

According to some embodiments of the invention, the AGI-134 is administered intratumorally.

According to some embodiments of the invention, the AGI-134 and said Pembrolizumab are administered once every three weeks for four cycles.

According to some embodiments of the invention, the Pembrolizumab is administered in the absence of said AGI-134 once every three weeks for up to one year of treatment.

According to some embodiments of the invention, the dose of AGI-134 per administration is between 25 mg-200 mg.

According to some embodiments of the invention, the dose of Pembrolizumab per administration is 100 mg 500 mg per administration.

According to some embodiments of the invention, the dose of Pembrolizumab per administration is 200 mg per administration.

According to some embodiments of the invention, the subject was treated previously to surgically remove the tumor.

According to some embodiments of the invention, the subject was not treated previously to surgically remove the tumor.

According to some embodiments of the invention, the tumor is a solid tumor.

According to some embodiments of the invention, the solid tumor is an unresectable metastatic solid tumor.

According to some embodiments of the invention, the tumor is a tumor originating from an organ selected from peritoneum, liver, pancreas, lung, urinary bladder, prostate, uterus, cervix, vagina, bone marrow, breast, skin, brain, lymph node, head and neck, stomach, intestine, colon, kidney, testis and ovaries.

According to some embodiments of the invention, the tumor comprises a primary tumor and/or a metastasis.

According to some embodiments of the invention, the tumor comprises melanoma, sarcoma, glioma, or carcinoma cells.

According to some embodiments of the invention, the subject has metastatic colorectal cancer.

According to some embodiments of the invention, the subject has squamous cell carcinoma of the head and neck.

According to some embodiments of the invention, the Pembrolizumab and said AGI-134 are co-formulated.

According to some embodiments of the invention, the Pembrolizumab and said AGI-134 are in separate formulations.

According to some embodiments of the invention, the Pembrolizumab is formulated for intravenous delivery.

According to some embodiments of the invention, the AGI-134 is formulated for intratumoral delivery.

According to some embodiments of the invention, the AGI-134 is provided in a unit dosage form selected from the group consisting of 25 mg, 50 mg, 100 mg, 150 mg and 200 mg.

According to some embodiments of the invention, the Pembrolizumab is provided in a unit dosage form of about 200 mg.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is the structural formula of AGI-134.

FIG. 2 illustrates the synthesis of AGI-134.

### DESCRIPTION OF SPECIFIC EMBODIMENTS OF THE INVENTION

The present invention, in some embodiments thereof, relates to a combination therapy for treatment of cancer.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

According to a first aspect of the present invention, there is provided a method of treating a tumor in a subject, comprising administering to the subject a therapeutically effective amount of:

i) Pembrolizumab; and

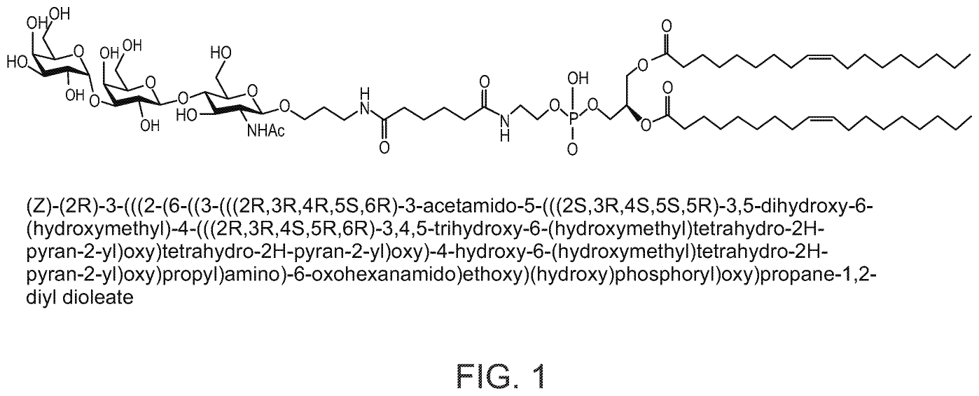

ii) a lipid having a structural formula as illustrated in FIG. 1 (AGI-134);

thereby treating the tumor in the subject.

The term “AGI-134”, also known as α-Gal BOEL” is an α-Gal bridged bis-octadecenoate lipid the structural formula of which is illustrated in FIG. 1. In one embodiment, it has the full chemical name (according to IUPAC convention) of (9Z,9′Z)-(2R)-3-(((2-(6-((3-(((2R,3R,4R,5S,6R)-3-acetamido-5-(((2S,3R,4S,-5S,6R)-3, 5-di hydroxy-6-(hydroxymethyl)-4-(((2R,3R,4S,5R,6R)-3,4,5-trihydroxy-6-(hydroxymethyl)tetrahydro-2H-pyran-2-yl)oxy)tetrahydro-2H-pyran-2-yl)oxy)-4-hydroxy-6-(hydroxymethyl)tetrahydro-2H-pyran-2-yl)oxy)propyl)amino)-6-oxohexana-mido)ethoxy)(hydroxy)pho sphoryl)oxy)propane-1,2-diyl bis(octadec-9-enoate). It has the chemical formula C70H125N3NaO26P and molecular weight: 1478.71. This molecule is disclosed in US Patent Application No. 20170266214.

AGI-134 is commercially available from Sigma-Aldrich under the product name ‘FSL-Galili(tri)™’ (Catalogue No. F9432). This construct consists of a functional (F), spacer (S) and lipid (L) component and can be used to insert into cell membranes so that the cell will display the functional (F) component on its surface. The functional component of AGI-134 is a trisaccharide group of: Gal-α1-3-Gal-β1-4GlcNAc (i.e. the α-Gal epitope). The spacer component is a O(CH₂)_{3NH} group and the lipid component is an adipate derivative (i.e. OOC(CH₂)_{4COO}, the ionized form of adipic acid) of dioleoylphosphatidylethanolamine (DOPE).

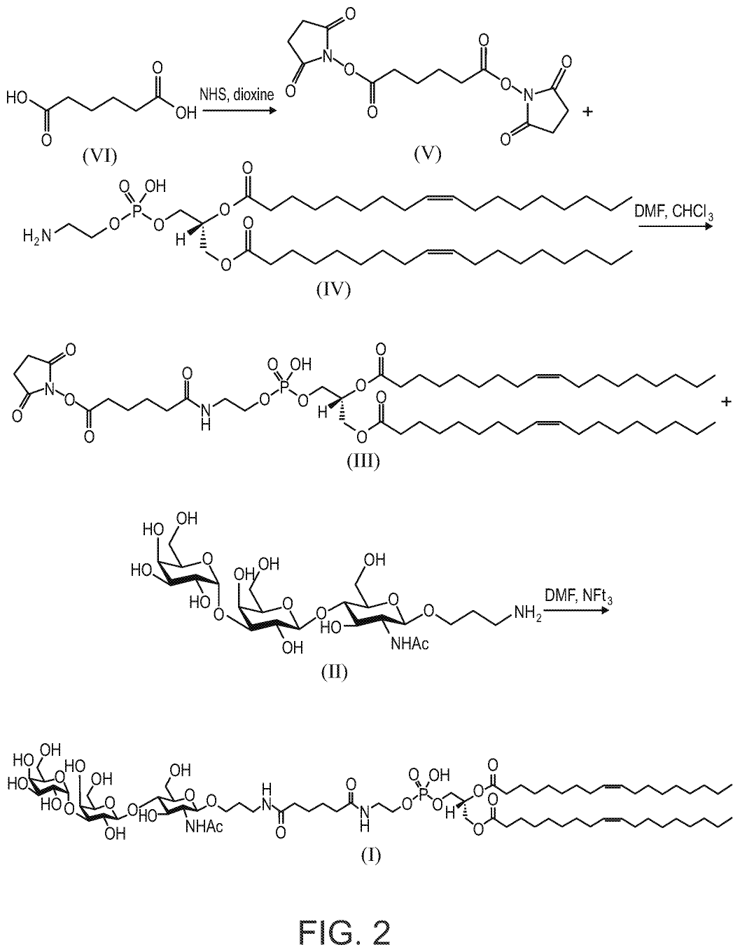

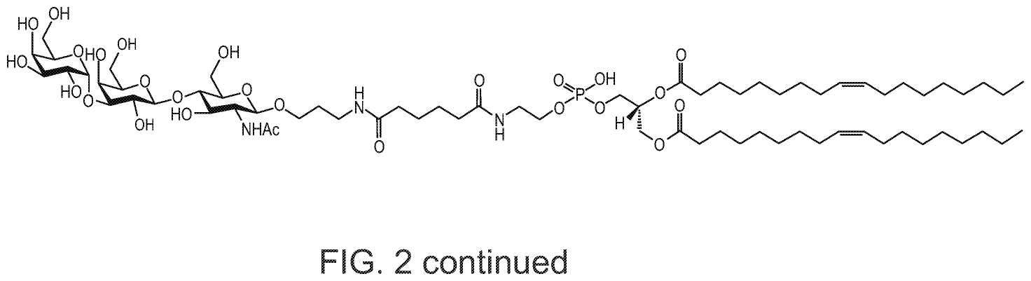

Synthesis of AGI-134 can be carried out as detailed in FIG. 2 and further detailed in Example 2 below.

The term “Pembrolizumab” refers to a humanized antibody that acts as a PD-1 checkpoint inhibitor (also known as MK-3475, Merck 3475, KEYTRUDA® (Merck Sharp & Dohme Corp., Whitehouse Station, N.J.) and SCH-900475.

The AGI-134 and the Pembrolizumab may be administered per se or as part of a pharmaceutical composition.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the Pembrolizumab or the AGI-134 accountable for the biological effect.

In one embodiment, the Pembrolizumab is formulated in a separate formulation to the AGI-134.

In another embodiment, the Pembrolizumab is co-formulated with AGI-134.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

Suitable routes of administration may, for example, include oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, or intraocular injections.

For buccal administration, the compositions may take the form of tablets or lozenges formulated in conventional manner.

For administration by nasal inhalation, the active ingredients for use according to some embodiments of the invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of some embodiments of the invention may also be formulated in rectal compositions such as suppositories or retention enemas, using, e.g., conventional suppository bases such as cocoa butter or other glycerides.

According to a particular embodiment, the Pembrolizumab is administered intravenously.

According to another embodiment, the AGI-134 is administered intratumorally.

Pharmaceutical compositions suitable for use in context of some embodiments of the invention include compositions wherein the active ingredients are contained in an amount effective to achieve the intended purpose. More specifically, a therapeutically effective amount means an amount of active ingredients (Pembrolizumab and AGI-134) effective to prevent, alleviate or ameliorate symptoms of a disorder (e.g., cancer) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide levels of the active ingredient are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

In one embodiment, the amount of AGI-134 provided (e.g. intratumorally) is between 25 mg-200 mg. Exemplary doses include, but are not limited to 25 mg, 50 mg, 75 mg, 100 mg, 125 mg, 150 mg, 175 mg or 200 mg per subject. The concentration of AGI-134 in the formulation is typically between 10-50 mg/ml for example 25 mg/ml. The total injected drug volume may be injected in one or more lesions and according to the longest dimension measured for lesions selected for injection and according to the guidelines presented in the table below. More than one lesion might be injected.

TABLE 1
Determination of AGI-134 Injection Volume Based on Lesion Size

| | AGI-134 injection |
|---|---|
| Longest tumor dimension (cm) | volume to be used (mL) |
| 1.5-2.5 | Up to 1.0 |
| >2.5-5 | Up to 2.0 |
| >5-10 | Up to 4.0 |
| >10 | Up to 8.0 |

When lesions are clustered together, they may be injected together as a single lesion according to Table 1, herein above.

An exemplary dose (e.g. intravenous dose) of Pembrolizumab is between 100-500 mg per administration (e.g. 200 mg).

The Pembrolizumab may be provided as a 30 minute i.v. infusion.

Depending on the severity and responsiveness of the condition to be treated, dosing can be of a single or a plurality of administrations, with course of treatment lasting from several days to several weeks or until cure is effected or diminution of the disease state is achieved.

In one embodiment, the AGI-134 and the Pembrolizumab are provided once a week.

In another embodiment, the AGI-134 and the Pembrolizumab are provided once every two weeks.

In another embodiment, the AGI-134 and the Pembrolizumab are provided once every three weeks.

In another embodiment, the AGI-134 and the Pembrolizumab are provided once every four weeks.

In another embodiment, the AGI-134 and the Pembrolizumab are provided once every five weeks.

In another embodiment, the AGI-134 and the Pembrolizumab are provided once every six weeks.

In another embodiment, the AGI-134 and the Pembrolizumab are provided once every seven weeks.

In another embodiment, the AGI-134 and the Pembrolizumab are provided once every eight weeks.

Typically, the AGI-134 and the Pembrolizumab are provided not more than 6 hours apart, 5 hours apart, 4 hours apart, three hours apart, two hours apart, 1 hour apart or even 30 minutes apart.

In a particular embodiment, the AGI-134 and the Pembrolizumab are co-administered.

In one embodiment, the AGI-134 is provided initially and the Pembrolizumab is provided thereafter.

In another embodiment, the Pembrolizumab is provided initially and the AGI-134 is provided thereafter.

The number of cycles of administration can include one, two, three, four, five, six, seven, eight, nine, ten or more. In a particular embodiment, the number of cycles of administration is four.

A typical dosing regimen is described herein below:

i) intratumoral administration of between 25-200 mg of AGI-134;

ii) intravenous administration of 200 mg of Pembrolizumab; wherein the Pembrolizumab and the AGI-134 are administered not more than two hours apart (preferably the Pembrolizumab is administered following the intratumoral administration of the AGI-134), wherein the Pembrolizumab and the AGI-134 are administered once every three weeks for a total of four cycles.

The present invention conceives of continuing the treatment with Pembrolizumab once the treatment with AGI-134 has been terminated (e.g. after three cycles of treatment). This treatment with Pembrolizumab may be continued for up to 1 year using the same treatment regimen (e.g. once every three weeks).

The amount of a composition to be administered will, of course, be dependent on the subject being treated, the severity of the affliction, the manner of administration, the judgment of the prescribing physician, etc.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

The term “subject”, as used herein, refers to any organism that is capable of developing a tumor. Such organisms include, but are not limited to, mammals, humans, non-primate mammals, prosimians and New World monkeys etc.

The term “tumor” as used herein, refers to an abnormal mass of tissue which results from an abnormal growth or division of cells. Such tumors may be solid (i.e. a mass of cells in particular organ, tissue or gland, such as on the peritoneum, liver, pancreas, lung, urinary bladder, prostate, uterus, cervix, vagina, breast, skin, brain, lymph node, head and neck, stomach, intestine, colon or ovaries) or non-solid (i.e. liquid tumors which develop in the blood, such as leukaemia).

In one embodiment, the tumor is a solid tumor, myeloma, or a lymphoma. In a further embodiment, the tumor is a solid tumor. In an alternative embodiment, the tumor is a non-solid tumor.

In one embodiment, the tumor is a tumor originating from an organ selected from peritoneum, liver, pancreas, lung, urinary bladder, prostate, uterus, cervix, vagina, bone marrow, breast, skin, brain, lymph node, head and neck, stomach, intestine, colon, kidney, testis, and ovaries. In a further embodiment, the tumor is a tumor originating from an organ selected from peritoneum, liver, pancreas, lung, urinary bladder, prostate, uterus, cervix, vagina, breast, skin, brain, lymph node, head and neck, stomach, intestine, colon and ovaries.

In one embodiment, the tumor comprises a primary tumor and/or a metastasis. In a further embodiment, the tumor comprises a primary tumor. In an alternative embodiment, the tumor comprises a secondary tumor.

In one embodiment, the tumor comprises melanoma, sarcoma, glioma, or carcinoma cells. In a further embodiment, the tumor comprises melanoma or carcinoma cells, or a metastasis.

According to a particular embodiment, the tumor is a nonresectable tumor.

The term “nonresectable”, as used herein, refers to any part of an organ or bodily structure that cannot be surgically removed. For example, a “nonresectable tumor” may be a tumor physically unreachable by conventional surgical techniques, a tumor where its removal does not improve the overall cancer disease or wellbeing of the patient, or a tumor where its removal may be detrimental to a vital organ.

In one embodiment, the subject was treated previously to surgically remove the tumor.

In an alternative embodiment, the subject was not treated previously to surgically remove the tumor, i.e., the method described herein may be performed as neo-adjuvant therapy several weeks prior to resection of the primary tumor.

In a particular embodiment the method is for treating metastatic colorectal cancer.

In another particular embodiment, the method is for treating squamous cell carcinoma of the head and neck.

For the treatment of a tumor, the compound of the invention may be advantageously employed in combination with one or more other medicinal agents, more particularly, with one or more anti-cancer agents or adjuvants (supporting agents in the therapy) in cancer therapy.

Examples of other therapeutic agents or treatments that may be administered together (whether concurrently or at different time intervals) with the compounds of the invention include but are not limited to: Topoisomerase I inhibitors; Antimetabolites; Tubulin targeting agents; DNA binder and topoisomerase II inhibitors; Alkylating Agents; Monoclonal Antibodies; Anti-Hormones; Signal Transduction Inhibitors; Proteasome Inhibitors; DNA methyl transferases; Cytokines and retinoids; Chromatin targeted therapies; Radiotherapy; and other therapeutic or prophylactic agents.

Particular examples of anti-cancer agents or adjuvants (or salts thereof), include but are not limited to any of the agents selected from groups (i)-(xlvi), and optionally group (xlvii), below: (i) Platinum compounds, for example cisplatin (optionally combined with amifostine), carboplatin or oxaliplatin; (ii) Taxane compounds, for example paclitaxel, paclitaxel protein bound particles (Abraxane™), docetaxel, cabazitaxel or larotaxel; (iii) Topoisomerase I inhibitors, for example camptothecin compounds, for example camptothecin, irinotecan (CPT11), SN-38, or topotecan; (iv) Topoisomerase II inhibitors, for example anti-tumour epipodophyllotoxins or podophyllotoxin derivatives for example etoposide, or teniposide; (v) Vinca alkaloids, for example vinblastine, vincristine, liposomal vincristine (Onco-TCS), vinorelbine, vindesine, vinflunine or vinvesir; (vi) Nucleoside derivatives, for example 5-fluorouracil (5-FU, optionally in combination with leucovorin), gemcitabine, capecitabine, tegafur, UFT, S1, cladribine, cytarabine (Ara-C, cytosine arabinoside), fludarabine, clofarabine, or nelarabine; (vii) Antimetabolites, for example clofarabine, aminopterin, or methotrexate, azacitidine, cytarabine, floxuridine, pentostatin, thioguanine, thiopurine, 6-mercaptopurine, or hydroxyurea (hydroxycarbamide); (viii) Alkylating agents, such as nitrogen mustards or nitrosourea, for example cyclophosphamide, chlorambucil, carmustine (BCNU), bendamustine, thiotepa, melphalan, treosulfan, lomustine (CCNU), altretamine, busulfan, dacarbazine, estramustine, fotemustine, ifosfamide (optionally in combination with mesna), pipobroman, procarbazine, streptozocin, temozolomide, uracil, mechlorethamine, methylcyclohexylchloroethylnitrosurea, or nimustine (ACNU); (ix) Anthracyclines, anthracenediones and related drugs, for example daunorubicin, doxorubicin (optionally in combination with dexrazoxane), liposomal formulations of doxorubicin (eg. Caelyx™, Myocet™, Doxil™), idarubicin, mitoxantrone, epirubicin, amsacrine, or valrubicin; (x) Epothilones, for example ixabepilone, patupilone, BMS-310705, KOS-862 and ZK-EPO, epothilone A, epothilone B, desoxyepothilone B (also known as epothilone D or KOS-862), aza-epothilone B (also known as BMS-247550), aulimalide, isolaulimalide, or luetherobin; (xi) DNA methyl transferase inhibitors, for example temozolomide, azacytidine or decitabine; (xii) Antifolates, for example methotrexate, pemetrexed disodium, or raltitrexed; (xiii) Cytotoxic antibiotics, for example antinomycin D, bleomycin, mitomycin C, dactinomycin, carminomycin, daunomycin, levamisole, plicamycin, or mithramycin; (xiv) Tubulin-binding agents, for example combrestatin, colchicines or nocodazole; (xv) Signal Transduction inhibitors such as Kinase inhibitors (e.g. EGFR (epithelial growth factor receptor) inhibitors, VEGFR (vascular endothelial growth factor receptor) inhibitors, PDGFR (platelet-derived growth factor receptor) inhibitors, MTKI (multi target kinase inhibitors), Raf inhibitors, mTOR inhibitors for example imatinib mesylate, erlotinib, gefitinib, dasatinib, lapatinib, dovotinib, axitinib, nilotinib, vandetanib, vatalinib, pazopanib, sorafenib, sunitinib, temsirolimus, everolimus (RAD 001), or vemurafenib (PLX4032/RG7204); (xvi) Aurora kinase inhibitors for example AT9283, barasertib (AZD1152), TAK-901, MK0457 (VX680), cenisertib (R-763), danusertib (PHA-739358), alisertib (MLN-8237), or MP-470; (xvii) CDK inhibitors for example AT7519, roscovitine, seliciclib, alvocidib (flavopiridol), dinaciclib (SCH-727965), 7-hydroxy-staurosporine (UCN-01), JNJ-7706621, BMS-387032 (a.k.a. SNS-032), PHA533533, PD332991, ZK-304709, or AZD-5438;] (xviii) PKA/B inhibitors and PKB (akt) pathway inhibitors for example AT13148, AZ-5363, Semaphore, SF1126 and MTOR inhibitors such as rapamycin analogues, AP23841 and AP23573, calmodulin inhibitors (forkhead translocation inhibitors), API-2/TCN (triciribine), RX-0201, enzastaurin HCl (LY317615), NL-71-101, SR-13668, PX-316, or KRX-0401 (perifosine/NSC 639966); (xix) Hsp90 inhibitors for example AT13387, herbimycin, geldanamycin (GA), 17-allylamino-17-desmethoxygeldanamycin (17-AAG) e.g. NSC-330507, Kos-953 and CNF-1010, 17-dimethylaminoethylamino-17-demethoxygeldanamycin hydrochloride (17-DMAG) e.g. NSC-707545 and Kos-1022, NVP-AUY922 (VER-52296), NVP-BEP800, CNF-2024 (BIIB-021 an oral purine), ganetespib (STA-9090), SNX-5422 (SC-102112) or IPI-504; (xx) Monoclonal Antibodies (unconjugated or conjugated to radioisotopes, toxins or other agents), antibody derivatives and related agents, such as anti-CD, anti-VEGFR, anti-HER2 or anti-EGFR antibodies, for example rituximab (CD20), ofatumumab (CD20), ibritumomab tiuxetan (CD20), GA101 (CD20), tositumomab (CD20), epratuzumab (CD22), lintuzumab (CD33), gemtuzumab ozogamicin (CD33), alemtuzumab (CD52), galiximab (CD80), trastuzumab (HER2 antibody), pertuzumab (HER2), trastuzumab-DM1 (HER2), ertumaxomab (HER2 and CD3), cetuximab (EGFR), panitumumab (EGFR), necitumumab (EGFR), nimotuzumab (EGFR), bevacizumab (VEGF), ipilimumab (CTLA4), catumaxumab (EpCAM and CD3), abagovomab (CA125), farletuzumab (folate receptor), elotuzumab (CS1), denosumab (RANK ligand), figitumumab (IGF1R), CP751,871 (IGF1R), mapatumumab (TRAIL receptor), metMAB (met), mitumomab (GD3 ganglioside), naptumomab estafenatox (5T4), or siltuximab (IL6); (xxi) Estrogen receptor antagonists or selective estrogen receptor modulators (SERMs) or inhibitors of estrogen synthesis, for example tamoxifen, fulvestrant, toremifene, droloxifene, faslodex, or raloxifene;] (xxii) Aromatase inhibitors and related drugs, such as exemestane, anastrozole, letrazole, testolactone aminoglutethimide, mitotane or vorozole; (xxiii) Antiandrogens (i.e. androgen receptor antagonists) and related agents for example bicalutamide, nilutamide, flutamide, cyproterone, or ketoconazole; (xxiv) Hormones and analogues thereof such as medroxyprogesterone, diethylstilbestrol (a.k.a. diethylstilboestrol) or octreotide; (xxv) Steroids for example dromostanolone propionate, megestrol acetate, nandrolone (decanoate, phenpropionate), fluoxymestrone or gossypol, (xxvi) Steroidal cytochrome P450 17alpha-hydroxylase-17,20-lyase inhibitor (CYP17), e.g. abiraterone; (xxvii) Gonadotropin releasing hormone agonists or antagonists (GnRAs) for example abarelix, goserelin acetate, histrelin acetate, leuprolide acetate, triptorelin, buserelin, or deslorelin; (xxviii) Glucocorticoids, for example prednisone, prednisolone, dexamethasone; (xxix) Differentiating agents, such as retinoids, rexinoids, vitamin D or retinoic acid and retinoic acid metabolism blocking agents (RAMBA) for example accutane, alitretinoin, bexarotene, or tretinoin; (xxx) Farnesyltransferase inhibitors for example tipifarnib; (xxxi) Chromatin targeted therapies such as histone deacetylase (HDAC) inhibitors for example sodium butyrate, suberoylanilide hydroxamide acid (SAHA), depsipeptide (FR 901228), dacinostat (NVP-LAQ824), R306465/JNJ-16241199, JNJ-26481585, trichostatin A, vorinostat, chlamydocin, A-173, JNJ-MGCD-0103, PXD-101, or apicidin; (xxxii) Proteasome Inhibitors for example bortezomib, carfilzomib, CEP-18770, MLN-9708, or ONX-0912; (xxxiii) Photodynamic drugs for example porfimer sodium or temoporfin; (xxxiv) Marine organism-derived anticancer agents such as trabectidin;] (xxxv) Radiolabelled drugs for radioimmunotherapy for example with a beta particle-emitting isotope (e.g., Iodine-131, Yittrium-90) or an alpha particle-emitting isotope (e.g., Bismuth-213 or Actinium-225) for example ibritumomab or Iodine tositumomab; (xxxvi) Telomerase inhibitors for example telomestatin; (xxxvii) Matrix metalloproteinase inhibitors for example batimastat, marimastat, prinostat or metastat; (xxxviii) Recombinant interferons (such as interferon-.gamma. and interferon .alpha.) and interleukins (e.g. interleukin 2), for example aldesleukin, denileukin diftitox, interferon alfa 2a, interferon alfa 2b, or peginterferon alfa 2b; (xxxix) Selective immunoresponse modulators for example thalidomide, or lenalidomide; (xl) Therapeutic Vaccines such as sipuleucel-T (Provenge) or OncoVex; (xli) Cytokine-activating agents include Picibanil, Romurtide, Sizofiran, Virulizin, or Thymosin; (xlii) Arsenic trioxide; (xliii) Inhibitors of G-protein coupled receptors (GPCR) for example atrasentan; (xliv) Enzymes such as L-asparaginase, pegaspargase, rasburicase, or pegademase; (xlv) DNA repair inhibitors such as PARP inhibitors for example, olaparib, velaparib, iniparib, INO-1001, AG-014699, or ONO-2231; (xlvi) Agonists of Death receptor (e.g. TNF-related apoptosis inducing ligand (TRAIL) receptor), such as mapatumumab (formerly HGS-ETR1), conatumumab (formerly AMG 655), PRO95780, lexatumumab, dulanermin, CS-1008, apomab or recombinant TRAIL ligands such as recombinant Human TRAIL/Apo2 Ligand; (xlvii) Prophylactic agents (adjuncts); i.e. agents that reduce or alleviate some of the side effects associated with chemotherapy agents, for example anti-emetic agents, agents that prevent or decrease the duration of chemotherapy-associated neutropenia and prevent complications that arise from reduced levels of platelets, red blood cells or white blood cells, for example interleukin-11 (e.g. oprelvekin), erythropoietin (EPO) and analogues thereof (e.g. darbepoetin alfa), colony-stimulating factor analogs such as granulocyte macrophage-colony stimulating factor (GM-CSF) (e.g. sargramostim), and granulocyte-colony stimulating factor (G-CSF) and analogues thereof (e.g. filgrastim, pegfilgrastim), agents that inhibit bone resorption such as denosumab or bisphosphonates e.g. zoledronate, zoledronic acid, pamidronate and ibandronate, agents that suppress inflammatory responses such as dexamethasone, prednisone, and prednisolone, agents used to reduce blood levels of growth hormone and IGF-I (and other hormones) in patients with acromegaly or other rare hormone-producing tumours, such as synthetic forms of the hormone somatostatin e.g. octreotide acetate, antidote to drugs that decrease levels of folic acid such as leucovorin, or folinic acid, agents for pain e.g. opiates such as morphine, diamorphine and fentanyl, non-steroidal anti-inflammatory drugs (NSAID) such as COX-2 inhibitors for example celecoxib, etoricoxib and lumiracoxib, agents for mucositis e.g. palifermin, agents for the treatment of side-effects including anorexia, cachexia, oedema or thromoembolic episodes, such as megestrol acetate.

In a further embodiment, the pharmaceutical composition additionally comprises one or more enhancers of immune system up-regulation. Examples of suitable enhancers of immune system up-regulation are described in US 2012/263677 and include suitable non-specific cytokines, such as interleukin-1, -2, or -6 (IL-1, IL-2 or IL-6) and aldesleukin; interferon-alpha or gamma (IFN-.alpha. and IFN-.gamma.), interferon alfa-2b and pegylated interferon (including pegylated interferon alfa-2a and pegylated interferon alfa-2b); granulocyte macrophage colony stimulating factor (GM-CSF, molgramostim or sargramostim); dendritic cell vaccines and other allogeneic or autologous therapeutic cancer vaccines, including intralesional vaccines containing an oncolytic herpes virus encoding GM-CSF (OncoVex™ or a plasmid encoding human leukocyte antigen-B7 and beta-2 microglobulin agent designed to express allogeneic MHC class I antigens (Allovectin-7™); and antibodies against specific tumour antigens. In a yet further embodiment, the one or more enhancers of immune system up-regulation are selected from IL-2 and interferon-gamma.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion. Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Materials and Methods

### Protocol:

1. AGI-134 −via IT injection. The therapy will be provided as one dose every three week (altogether four treatments).
2. AGI-134+Pembrolizumab—injection. The combined therapy will be provided as one dose every three week (altogether four treatments). Pembrolizumab will continue to be administered IV for a total of up to 17 cycles (one year of treatment).

### Criteria: Inclusion Criteria

1. Adult male or female aged 18 years old or older.
2. With a histologically—or cytologically—confirmed unresectable metastatic solid tumor and who have received, or been intolerant to, all treatment options known to confer clinical benefit.
3. Subjects should have at least two measurable lesions based on RECIST v1.1 as determined by the site study team.
4. Subjects who are willing to undergo tumor biopsies, unless tumor is considered inaccessible or biopsy is otherwise considered not in the subject's best interest.
5. With sufficient tumor size for IT injection.
6. Has ≥1 injectable lesion which is amenable to injection and biopsy ads measurable according to RECIST v1.1.
7. Has ≥1 metastatic lesion which is amenable for biopsy.

8. Evaluable Disease according to RECIST v1.1.
9. Has an Eastern Cooperative Oncology Group (ECOG) Performance Status score of 0 or 1.
10. Has a life expectancy >3 months.
11. Adequate organ function.

12. Women of childbearing potential and all men must agree to use an adequate contraception.
13. Subject is able and willing to comply with the requirements of the protocol.
14. Subject is able to voluntarily provide written informed consent.

### Exclusion Criteria:

1. Has a disease that is suitable for therapy administered with curative intent.
2. Has any active, acute, or chronic infection(s) that are uncontrolled and/or requiring treatment, such as antibiotics.
3. An active autoimmune disease that has required systemic treatment in the 2 years preceding the study.
4. History of or plan for splenectomy or splenic irradiation.
5. History of organ transplant or currently taking active immunosuppressive therapy.
6. Has a known history of Human Immunodeficiency Virus (HIV) (HIV 1/2 antibodies).
7. Has known active or chronic Hepatitis B or Hepatitis C.
8. History or evidence of cancer associated with immunodeficiency states.
9. Has a diagnosis of immunodeficiency or is receiving systemic steroid therapy or any other form of immunosuppressive therapy within 7 days prior to the first dose of trial treatment.
10. Is expected to require any other form of antineoplastic therapy while on study.
11. Had received live vaccines within 30 days prior to the first dose of trial treatment.
12. Has positive IgE anti Gal.
13. Subject has a known allergy to alpha-Gal, such as red meat allergy, exposure to lone star tick (Amblyomma americanum), Ixodes ricinus/holocyclus, or Cetuximab allergy.
14. Has known allergy or hypersensitivity to any of the test compounds, materials or contraindication to test product.
15. History or evidence of central nervous system metastases and/or carcinomatous meningitis (unless stable without treatment for at least 6 weeks and not requiring steroids).
16. Has received other experimental therapies or used an investigational device within 28 days of the first dose of treatment.
17. Has had prior chemotherapy, targeted small molecule therapy, or radiation therapy within 14 days prior to study Day 1 or has not recovered from AE≤Grade 1 by treatment administered more than 14 days before first dose.
18. Has had a prior anti-cancer monoclonal antibody (mAb) within 28 days prior to study Day 1 or who has not recovered from AE≤Grade 1 by treatment administered more than 28 days earlier.
19. Is pregnant or breastfeeding or expecting to conceive or father children within the projected duration of the trial, starting with the screening visit through 120 days after the last dose of trial treatment.
20. Has unstable angina, new onset angina within the last 3 months, myocardial infarction within the last 6 months, uncontrolled atrial fibrillation, or current congestive heart failure with New York Heart Association Class III or higher.
21. Has a known current additional malignancy that is progressing or requires active treatment.
22. O₂ saturation <92% (on room air).
23. Has an underlying medical condition that would preclude study participation or other psychological, social or physical examination finding or a laboratory abnormality that the Investigator considers would make the subject a poor trial candidate or could interfere with protocol compliance or the interpretation of trial results.
24. Has known psychiatric or substance abuse disorders that would interfere with cooperation with the requirements of the trial.
25. Has a history of (non-infectious) pneumonitis that required steroids or current pneumonitis.
26. Has a history of interstitial lung disease.

AGI-134

In a dose escalation study, the total dose will increment while the concentration will be kept constant at 25 mg/ml, by increasing the total volume injected. A single subject will be administered per dose level beginning with a dose of 25 mg (1 ml) and increasing by 100% to 50 mg (2 ml), to 100 mg (4 ml) and up to a maximal dose of 200 mg (8 ml). The total injected drug volume will be injected in one or more lesions and according to the longest dimension measured for the superficial and/or palpable lesions selected for injection. More than one lesion might be injected according to the dose and volume at each escalation level.

Subjects will be administered with one dose per cycle of AGI-134 monotherapy, every three weeks, for up to 4 cycles.

In the expansion part of the study, subjects will be enrolled and receive intratumoral injection according to their lesion size as described in Table 1 above.

Pembrolizumab

Treatment with pembrolizumab will be part of the combination therapy with AGI-134. During the combination period, Pembrolizumab will be administered as a dose of 200 mg using a 30-minute IV infusion on Day 1 of each cycle after all procedures and assessments have been completed. Pembrolizumab should be administered 1 hour (−5 min/+10 min) after AGI-134 injection.

### Example 2

### Synthesis of AGI-134

To a solution of 3-aminopropyl 4-O-[3-O-(α-D-Galactopyranosyl)-β-D-Galactopyranosyl]-2-acetam-ido-2-deoxy-β-D-glucopyranoside (II) (Mendeleev Communications, 2002, (143-145) or Tetrahedron, 61, (2005), 4313-4321, 52 mg, 0.086 mmol) in dry DMF (2 mL) was added 15 μL, of Et₃N followed by a solution of DOPE-Ad-ONSu (III) (U.S. Pat. No. 8,013,131 B2, 100.6 mg, 1.00 mmol) in CH₂Cl₂ (2 mL). The reaction was stirred for 2 hours at room temperature followed by sequential column chromatography (the first on Sephadex LH-20, and the second on silica gel eluting with CH₂Cl₂-EtOH—H₂O; 6:5:1) to afford the title compound (1) (105.6 mg, 84%).

R_{f}0.5 (CH₂Cl₂-EtOH—H₂O; 6:5:1)
¹H NMR (700 MHz, CDCl₃-CD₃OD 1:1, 30° C.), δ, ppm, selected: 5.45-5.54 (m, 4H, 2×—CH—CH—), 5.34-5.43 (m, 1H, —OCH₂—CHO—CH₂O—) 5.18 (d, 1H, J_{1,2} 2.52, H-1^{m}) 4.61 (d, 1H, J_{1,2} 7.57, H-1ⁿ), 4.60 (dd, 1H, J 2.87, J 12.00, C(O)OCHHCHOCH₂O—), 4.56 (d, 1H, J 8.39, H-1^{I}), 4.36 (dd, 1H, J 6.8, J 12.00, —C(O)OCH HCHOCH₂O—), 4.19, (d, 1H, J_{3,4} 2.48, H-4^{II}), 4.13-4.18 (m, 2H, —CHO—CH₂OP—), 3.52-3.62 (m, 3H, PO—CH₂—C H₂—NH, —CH₂—CHH—NH), 3.29-3.35 (m, 1H, —CH₂—CHH—NH), 2.45-2.52 (m, 4H, 2×—CH₂—CO), 2.36-2.45 (m, 4H, 2×—CH₂—CO), 2.14-2.22 (m, 11H, 2×(—CH₂—CH—CH—CH₂—), NHC(O)CH₃), 1.85-1.96 (m, 2H, O—CH₂CH₂—NH) 1.73-1.84 (m, 8H, COCH₂CH₂CH₂CH₂CO and 2×(COCH₂CH₂—), 1.36-1.55 (m, 40H, 20CH₂), 1.05 (t, 6H, J 6.98, 2CH₃),
C70H126N3O26P; MALDI MS: m/z 1480 (M Na+H); 1496 (MK+H): 1502 (MNa+Na), 1518 (M Na+K)

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference.

In addition, citation or identification of any reference in this application shall not be construed as an admission that such reference is available as prior art to the present invention. To the extent that section headings are used, they should not be construed as necessarily limiting.

1. A method of treating a tumor in a subject, comprising administering to the subject a therapeutically effective amount of:
i) Pembrolizumab; and
ii) a lipid having a structural formula as illustrated in FIG. 1 (AGI-134);
thereby treating the tumor in the subject.

2. The method of claim 1, wherein said Pembrolizumab and said AGI-134 are in separate formulations.

3. The method of claim 2, wherein said Pembrolizumab is administered following administration of said AGI-134.

4. The method of claim 1, wherein said Pembrolizumab and said AGI-134 are administered not more than two hours apart.

5. The method of claim 1, further comprising administering to said subject Pembrolizumab in the absence of said AGI-134 following said administering of Pembrolizumab and said AGI-134.

6. The method of claim 1, wherein said Pembrolizumab is administered intravenously or intratumorally.

7. (canceled)

8. The method of claim 1, wherein said AGI-134 and said Pembrolizumab are administered once every three weeks for four cycles.

9. The method of claim 5, wherein said Pembrolizumab is administered in the absence of said AGI-134 once every three weeks for up to one year of treatment.

10. The method of claim 1, wherein a dose of AGI-134 per administration is between 25 mg-200 mg.

11. The method of claim 1, wherein a dose of Pembrolizumab per administration is 100 mg 500 mg per administration.

12. The method of claim 1, wherein a dose of Pembrolizumab per administration is 200 mg per administration.

13. A method of treating a tumor in a subject, comprising:
i) intratumorally administering to the subject between 25-200 mg of AGI-134;
ii) intravenously administering to the subject 200 mg of Pembrolizumab; wherein said Pembrolizumab and said AGI-134 are administered not more than two hours apart, wherein said Pembrolizumab and said AGI-134 are administered once every three weeks for a total of four cycles; and subsequently
(iii) intravenously administering to the subject 200 mg of Pembrolizumab once every three weeks for up to one year in the absence of said AGI-134, thereby treating the tumor in the subject.

14. The method of claim 1, wherein the subject was treated previously to surgically remove the tumor.

15. The method of claim 1, wherein the subject was not treated previously to surgically remove the tumor.

16. The method of claim 1, wherein said tumor is a solid tumor or an unresectable metastatic solid tumor.

17. (canceled)

18. The method of claim 1, wherein the tumor is a tumor originating from an organ selected from the group consisting of peritoneum, liver, pancreas, lung, urinary bladder, prostate, uterus, cervix, vagina, bone marrow, breast, skin, brain, lymph node, head and neck, stomach, intestine, colon, kidney, testis and ovaries.

19. The method of claim 1, wherein the tumor comprises a primary tumor and/or a metastasis.

20. The method of claim 1, wherein the tumor comprises melanoma, sarcoma, glioma, or carcinoma cells.

21. The method of claim 1, wherein the subject has metastatic colorectal cancer.

22. The method of claim 1, wherein the subject has squamous cell carcinoma of the head and neck.

23-37. (canceled)
